(19) United States
(12) Patent Application Publication
(10) Pub. No.: US 2014/0228732 A1
(43) Pub. Date: Aug. 14, 2014

(54) PNEUMATIC COMPRESSION BANDAGE

(71) Applicant: RevMedx, Inc., Wilsonville, OR (US)

(72) Inventors: John Steinbaugh, Wilsonville, OR (US); Paul Litchfield, Westborough, MA (US); Paul Davis, Blackstone, MA (US)

(73) Assignee: RevMedx, Inc., Wilsonville, OR (US)

(21) Appl. No.: 14/176,920

(22) Filed: Feb. 10, 2014

Related U.S. Application Data
(60) Provisional application No. 61/837,465, filed on Jun. 20, 2013.
(60) Provisional application No. 61/762,471, filed on Feb. 8, 2013.

(51) Int. Cl. A61F 13/00

(52) U.S. Cl. CPC A61F 13/00021; A61F 13/00063; A61F 2013/00106; A61F 2013/00119; A61F 2013/0028
USPC 602/53; 604/385.01

(57) ABSTRACT

Disclosed herein are compression bandages that include one or more fluid bladders (such as an air bladder) disposed in or on a bandage, such as an elastic bandage. In various embodiments, the one or more bladders may be adapted to apply focused pressure to a wound site, for example where direct pressure is required in order to stop or slow bleeding. In various embodiments, such a fluid bladder may permit sufficient pressure to be applied where needed, while also preventing the over-application of pressure to other tissues in proximity to the wound.

### CROSS REFERENCE TO RELATED APPLICATIONS

The present application claims priority to U.S. Provisional Patent Application No. 61/762,471, filed Feb. 8, 2013, entitled “PNEUMATIC COMPRESSION BANDAGE,” and to U.S. Provisional Patent Application No. 61/837,465, filed Jun. 20, 2013, entitled “PNEUMATIC COMPRESSION BANDAGE,” the disclosures of which are hereby incorporated by reference in their entirety.

### GOVERNMENT INTERESTS

This invention was made with government support under Contract No. W911 NF-11-C-0038 awarded by the Department of Defense. The government has certain rights in the invention.

### TECHNICAL FIELD

Embodiments herein relate to methods, compositions and devices for controlling bleeding and treating wounds.

### BACKGROUND

Uncontrolled external hemorrhage is the leading cause of death on the battlefield and the second leading cause of death in civilian trauma. At the point of injury, every drop of blood is precious and cannot be replaced until the casualty reaches the next echelon of care. Wounds sustained from IED's, blasts, and small caliber missiles can cause open and penetrating wounds that are difficult to pack with standard and hemostatic gauzes. Application of sufficient pressure on bleeding vessels to control hemorrhage is a constant challenge in austere environments. Elastic fabric wraps are often used as an outer layer of wound dressings to hold the wound contact and absorbent materials in place and provide compressive force to the wound. The use of fabric elastomeric wraps, however, is not always effective because these wraps generally distribute uniform circumferential pressure to a patient's limb, which may result in insufficient focal compression being applied to the wound.

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings.

FIG. 1 is an illustration of one example of a compression bandage having a pneumatic element, shown in a partially unrolled state, in accordance with various embodiments;

FIG. 2 is an illustration of another example of a compression bandage, shown in a rolled state, in accordance with various embodiments;

FIG. 3 is an illustration of another example of a compression bandage, shown with a compression bulb attached, in accordance with various embodiments;

FIGS. 4A and 4B are illustrations another example of a compression bandage, shown with a pressure indicator in an “unpopped” state (FIG. 4A) and a “popped” state (FIG. 4B), in accordance with various embodiments; and

FIG. 5 is a diagram of another example of a compression bandage having a bladder, a pressure indicator, and an inflation valve, for use in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Embodiments herein provide compression bandages that include one or more fluid bladders (such as an air bladder) disposed in or on a bandage. In various embodiments, the one or more bladders may be adapted to apply focused pressure to a wound site, for example where direct pressure is required in order to stop or slow bleeding. In various embodiments, such a fluid bladder may permit sufficient pressure to be applied where needed, while also preventing the over-application of pressure to other tissues in proximity to the wound. In some embodiments, the compression bandages may be wrapped into a roll or z-folded for easy storage. In some embodiments, the compression bandages may have a predetermined size and shape sufficient to cover an afflicted wound area.

Other embodiments are methods for treating hemorrhagic injuries. More specifically, methods are provided to effect rapid hemostatic response and control hemorrhage by applying a compression bandage having a fluid bladder to a bleeding wound, and inflating the fluid bladder to apply additional compression to a selected area on or around the wound.

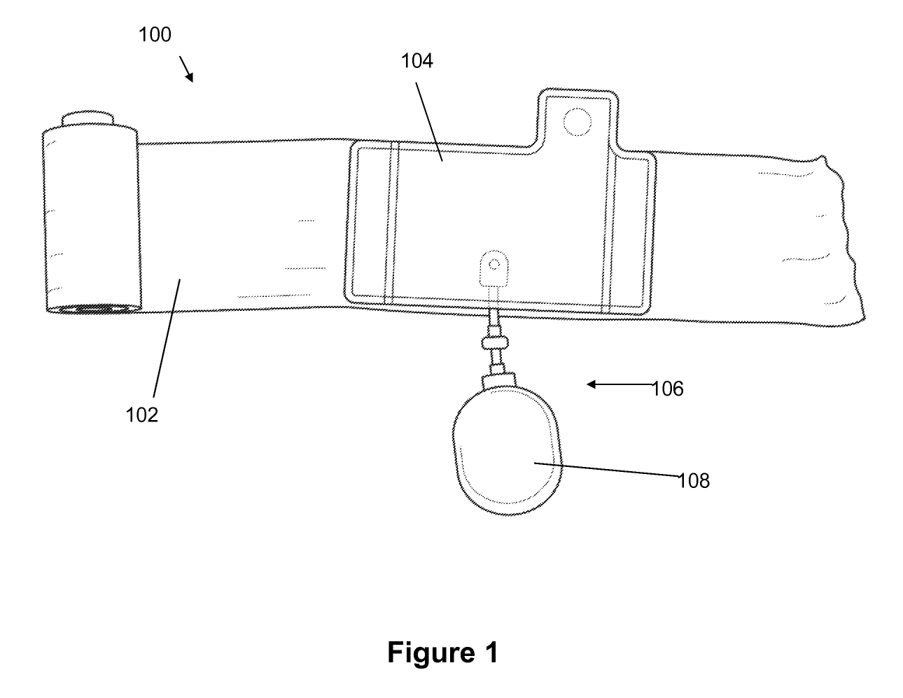

FIG. 1 is a diagram of one example of a compression bandage having a pneumatic element, shown in a partially unrolled state. As shown in FIG. 1, compression bandage 100 may include one or more fluid bladders, such as air bladder 104, disposed on a bandage 102. In some embodiments, bandage 102 may be an elastic bandage, such as a fabric wrap or elastic (such as an Ace™) bandage. In other embodiments, bandage 102 may be a self-adhering bandage.

In various embodiments, the one or more fluid bladders may be made from or include rubber and/or another natural or synthetic elastomeric material. In other embodiments, the one or more fluid bladders may include or be made from sheets of polyurethane or polyester film that are attached together to form an airtight seal. Alternatively, the one or more fluid bladders may be formed from a polyester film, such as MYLAR®, or a laminate, such as a film and cloth laminate.

In various embodiments, the compression bandage also may include an inflation mechanism 106 for selectively introducing air or another fluid into the air bladder 104. In some embodiments, inflation mechanism 106 may be small, lightweight, and may provide a sufficient volume of air such that only little effort is needed for adequate inflation. In various embodiments, inflation mechanism 106 also may include a compressible bulb 108, for example for forcing air into air bladder 104.

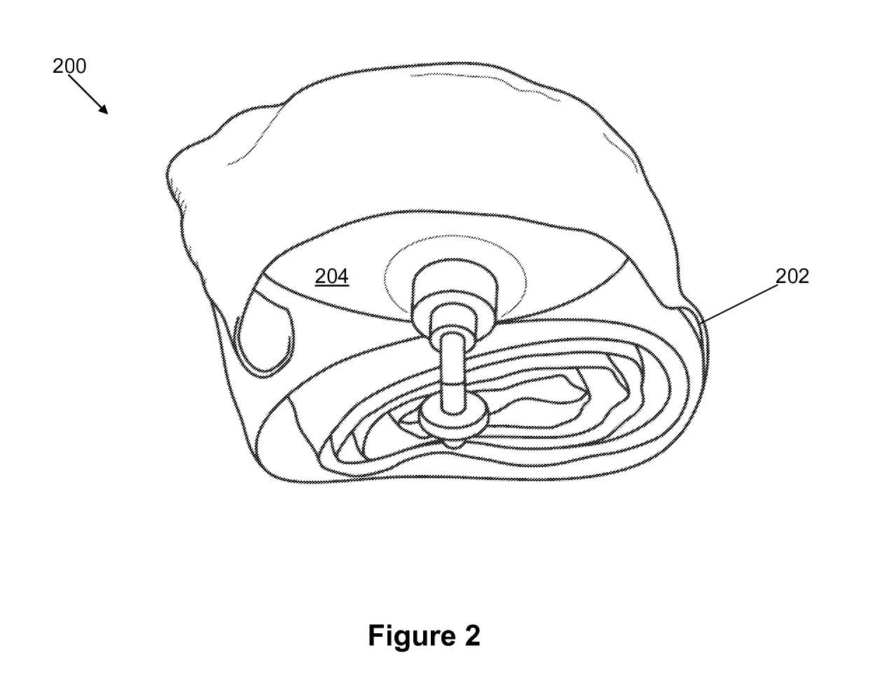

FIG. 2 is an illustration of another example of a compression bandage 200, shown with the bandage 202 in a rolled state with an inflated air bladder 204, in accordance with various embodiments. Although the embodiment illustrated in FIG. 2 includes an air bladder 204, one of skill in the art will appreciate that a fluid bladder may be filled with any suitable liquid or gas, and that as used herein, the term “fluid bladder” refers to bladder intended to hold a suitable liquid, such as water, saline, oil, or alcohol, or any suitable gas, such as air, oxygen, carbon dioxide, or helium. One of skill in the art will also appreciate that although the air bladder illustrated in FIG. 2 is configured to be filled with air or any other ambient gas, other embodiments may be filled with other, non-ambient gases or liquids, for example from a reservoir.

In various embodiments, air bladder 204 may take the form of one or more predetermined shapes, based on the desired length, width, diameter, or cross-sectional shape for a particular application. Without being bound by theory, the shape, size and/or pattern of the air bladder may influence the ability of the compression bandage 100 to fit into, expand, fill, partially fill and/or conform to a wound or wound cavity.

In various embodiments, air bladder 204 may include one or more welds to control the thickness of the air bladder 204 when the air bladder 204 is in its filled configuration (e.g., when air has been pumped into the air bladder 204). In various embodiments, the one or more welds may be formed by RF welding, heat welding, ultrasonic welding, or by other suitable means. In various embodiments, in regions of the air bladder 204 where it is desirable to have air bladder 204 inflated to a minimal thickness, the density or size of the welds may be greater than in the areas where it is permissible or desirable for air bladder 204 to be inflated to a greater thickness. In various embodiments, these welds may be circular, or any other geometry, such as linear, triangular, oval, or square, provided that they are shaped to limit and control the inflation dimensions of the air bladder 204 of the present disclosure.

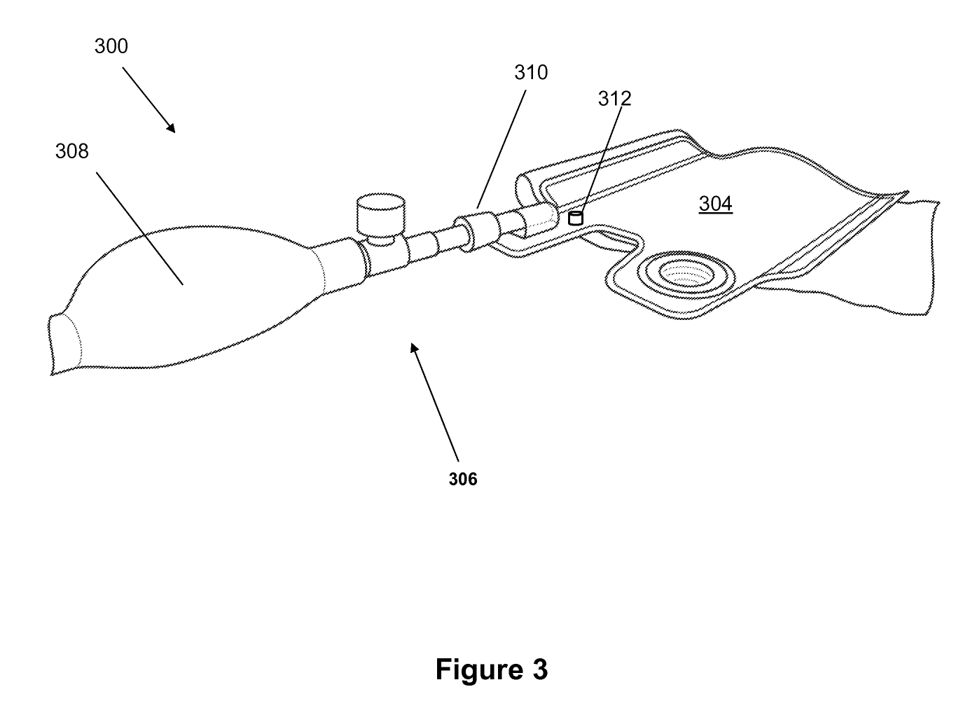

FIG. 3 is an illustration of another example of a compression bandage 300, shown with a compression bulb 308 attached, in accordance with various embodiments. In various embodiments, inflation mechanism 306 may include a handheld compressible bulb 308 (which may take any of various shapes known to those of skill in the art) with a one-way check valve. Thus, in various embodiments, when the compressible bulb 308 is compressed, such as by hand operation of user, air within the compressible bulb 308 may be forced into the air bladder 304. In various embodiments, as the compressible bulb 308 is released, the check valve opens because of the pressure void in the compressible bulb 308, allowing ambient air to enter the bulb.

In other embodiments, inflation mechanism 306 may include a manually collapsible vessel wherein the intake of fluid and the outflow of fluid can be managed with a variety of techniques (such as one way valves, including, but not limited to duckbill, umbrella, poppet, thin film, diaphragm, ball and also with physical impedance on an open port). Other embodiments may use an inflation mechanism having collapsible walls, in order to displace a greater volume of air. Other inflation mechanisms may include a temporarily collapsible foam insert within a compressible bulb. In these embodiments, the foam insert may ensure that when the compressible bulb is released, the compressible bulb expands to the natural volume of the foam insert, drawing in air to fill that volume. In such embodiments, the foam insert may include polyurethane foam. In other embodiments, the inflation mechanism may include an accordion style inflation mechanism that includes a plastic, collapsible case. In such embodiments, air enters through a port that is open to the exterior of the inflation mechanism. In various embodiments, the inflation mechanism may operate similarly to that described above with respect to the compressible bulb inflation mechanism, except that the casing may be collapsed accordion-style to increase the amount of air forced into the system. Upon release, the accordion-style casing may expand, and the air may be forced into the casing to regulate the pressure within the casing.

In some embodiments, inflation mechanism 306 may be adapted to have a certain pressure output to limit the amount of fluid that can be pumped into air bladder 304 and, thus, avoid over inflation of air bladder 304. Additionally, in various embodiments, a one-way valve 310 may be placed between inflation mechanism 306 and air bladder 304, so that once fluid enters the air bladder 304, it may not travel backwards into the inflation mechanism 306. Various types of one-way valves are suitable for use in conjunction with various inflation mechanisms in accordance with embodiments herein. In some embodiments, the one-way valve may be relatively small and flat for less bulkiness.

In various embodiments, the one-way valve 310 may provide a mechanism that helps avoid over-inflation of air bladder 304. In particular embodiments, if the pressure in the air bladder 304 is equal to the pressure exerted by the inflation mechanism 306, no additional air will be allowed to enter the air bladder 304. In some embodiments, when equilibrium is reached between the pressure in the air bladder 304 and the pressure of the compressed inflation mechanism 306, the one-way valve 310 that opens to allow fluid movement from the inflation mechanism 306 to the air bladder 304 may remain closed. In various embodiments, even if the one-way valve 310 does open, no more fluid will enter the air bladder 304. In addition, although a particular one-way valve 310 is illustrated, any suitable one-way valve would be appropriate for use in any of the embodiments disclosed herein.

In various embodiments, compression bandage 300 also may include a release valve 312, which may communicate between air bladder 304 and the ambient atmosphere. Thus, in various embodiments, with release valve 312, a user can reduce the amount of fluid in air bladder 304 manually. In various embodiments, the release valve 312 may be any suitable type of release valve, such as plunger, ball, umbrella, poppet, twist, button release, pinch, or fold outlet. For example, release valve 312 may be a plunger-type valve, wherein the fluid is released upon depression of a plunger that pushes a seal away from the wall of the air bladder 304 allowing fluid to escape. In particular embodiments, a release valve 312 may have a spring that biases a plunger in a closed position. In various embodiments, to release fluid from air bladder 304, the plunger may be depressed by the user. Fluid (such as air) then may escape around the sealing interface. In various embodiments, such a release valve may be mechanically simple and light weight. In various embodiments, the components of a release valve may be made out of a number of different materials, including plastic, elastomerics or metal.

In various embodiments, compression bandage 300 also may include a check valve, whereby the pressure in the air bladder 304 is automatically released. In other embodiments, a combination check valve and release valve maybe used. In various embodiments, a combination check valve and release valve may be made from a cap, a seating forming an air-tight seal with the cap, and a check valve forming another air-tight seal with the seating. In various embodiments, downward pressure on the cap may cause a separation between the seating and the cap, releasing the airtight seal between the cap and the seating.

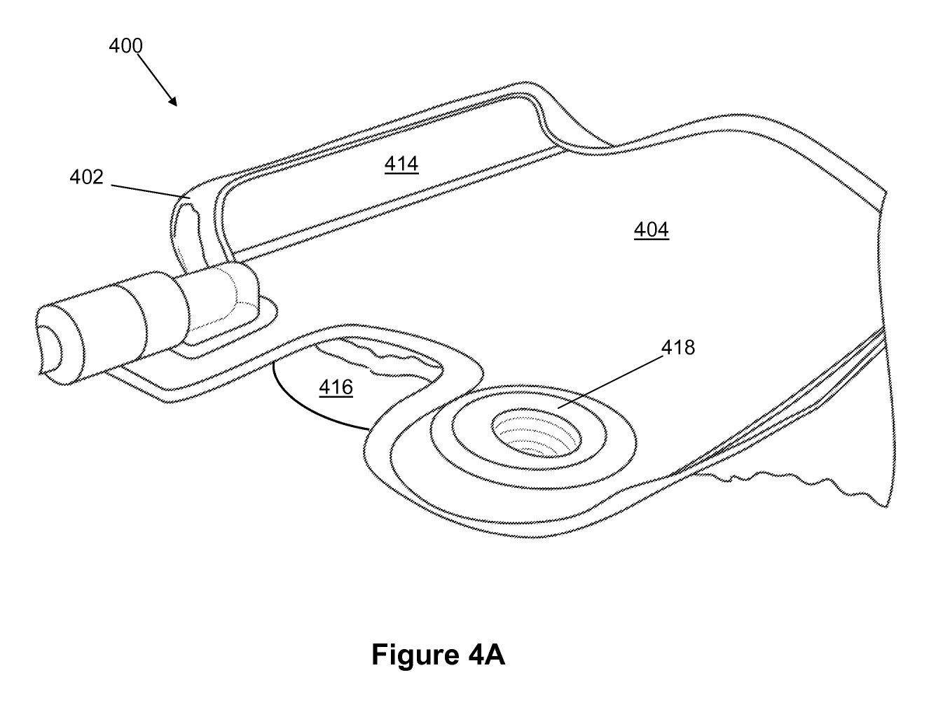

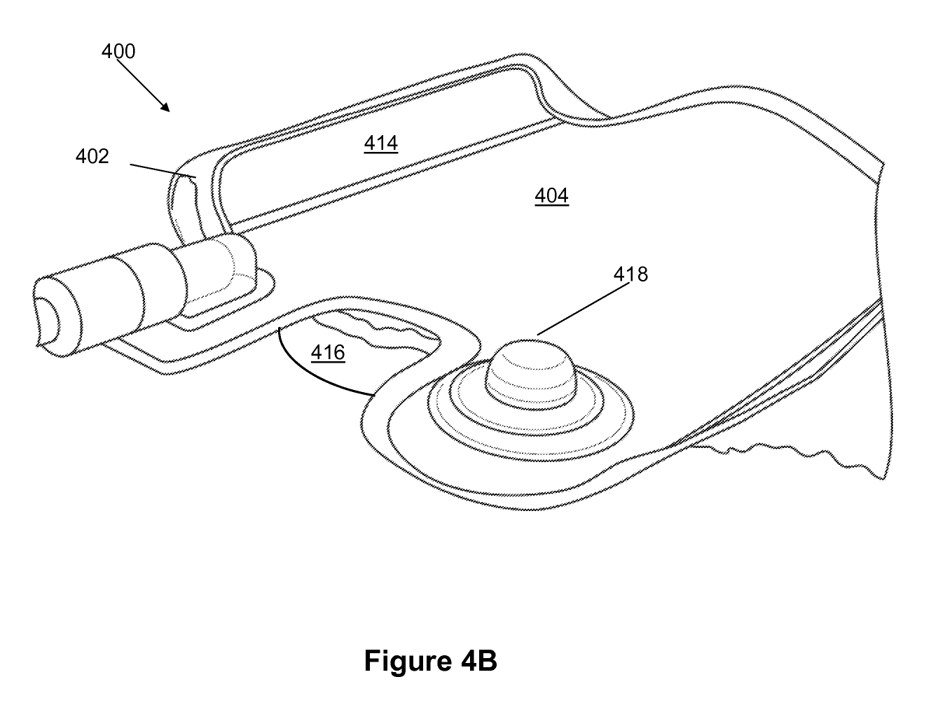

FIGS. 4A and 4B are illustrations another example of a compression bandage 400. In various embodiments, the air bladder 404 may be disposed on the bandage 402 by gluing, sewing, bonding, RF welding, heat welding, ultrasonic welding, or another method known to one skilled in the art, or with reversible mechanisms such as snaps, hook and loop closures, etc. Exemplary adhesives for adhering air bladder 404 to bandage 402 include cyanoacrylates, epoxies, light cure adhesives, silicones, urethanes, hydrogels, acrylics, silicone gels, silicone PSA and hydrocolloids. In various embodiments, air bladder 404 may be heat bonded to bandage 402. In such embodiments, air bladder 404 may be bonded to bandage 402 using a melted polypropylene layer. In other embodiments, air bladder 404 may be disposed on bandage 402 with stitching. In such embodiments, the stitching may be placed in a stitching margin 414, which may be formed when air bladder 404 is formed, wherein the stitching margin 414 is exterior to and adjacent to the bladder cavity, to permit stitching without piercing the cavity.

In various embodiments, bandage 402 may be in the form of a laminate dressing having a plurality of layers. In some embodiments, one or more of the layers may be joined to form a cell or pocket to receive air bladder 404 therein. In some embodiments, the cell or pocket may form a predetermined pattern or shape. In particular embodiments, the cell or pocket may be formed by adhering together two or more layers. In other embodiments, the cell or pocket may be formed by stitching together two or more layers.

In various embodiments, compression bandage 400 may include a wound-contacting absorbent layer 416. In various embodiments, a variety of materials may be used as the wound-contacting absorbent layer 416, including foams and woven or nonwoven materials, such as rayon, polyester, polyurethane, polyolefin, cellulose, cellulose derivatives, cotton, orlon, nylon, and hydrogel polymeric materials. In various embodiments, other types of materials having similar absorbent properties and characteristics may also be suitable for use in embodiments herein. In one specific embodiment, a wound-contacting absorbent layer 416 may be a woven or nonwoven material of natural or synthetic fibers made from, but not limited to, rayon, polyester, polyurethane, polyolefin, cellulose, cellulose derivatives, cotton, orlon, nylon, or hydrogel polymeric materials. Examples of suitable absorbent materials include dacron-polyester cast padding MW04, (3M, St. Paul, Minn.), DELTA-ROL™ acrylic cast padding 6884, (Johnson & Johnson, New Brunswick, N.J.), SOF-ROL™ 100% needle-tacked rayon cast padding HRI 8137-009034, (Johnson & Johnson, New Brunswick, N.J.), SPECIALIST™ cotton cast padding HRI 8137-009044 (Johnson & Johnson, New Brunswick, N.J.), WEBRIL™ cotton undercast padding 3175 (The Kendall Company, Boston, Mass.), WEBRIL II™ cotton undercast padding 4221 (The Kendall Company, Boston, Mass.), nonwoven cotton web 142-451 and nonwoven rayon/polyester web 140-037 (VeraTec Company, Wapole, Mass.), and an absorbent resilient open-cell foam such as polyurethane, polyester, polyether, polyolefin foams.

Other suitable absorbent materials include composite materials such as nonwoven polymeric matrices combined with highly hydrophilic fluid absorbing materials, and highly hydrophilic fluid absorbing materials include polymeric absorbent fibers or particles selected from the group consisting of modified polysaccharides, modified polyurethanes, and high molecular weight acrylic polymers containing hydrophilic groups.

In various embodiments, the wound-contacting absorbent layer 416 may include one or more layers of a nonwoven, melt-blown absorbent fiber which provides loft to the material and which-absorbs liquids. In various embodiments, the surface of the wound-contacting absorbent layer 416 that contacts the wound may additionally be treated or modified so that it will not adhere to the wound. For example, the wound-contacting absorbent layer 416 may be covered with a variety of commercially available wound contact materials such as TEGAPORE™ woven nylon web, TEGADERM™ polyurethane film or TEGASORB™ hydrocolloid (all available from 3M, St. Paul, Minn.) as well as other well-known related materials. In specific embodiments, the absorbent material of the wound-contacting absorbent layer 416 may be selected so that it will not inhibit the expansion of air bladder 404.

In various embodiments, the wound-contacting absorbent layer 416 may be liquid-expandable. For example, in some embodiments, the wound-contacting absorbent layer 416 may expand in occupied volume upon contact with a liquid. The liquid may be an aqueous solution, such as a bodily fluid. For example, the liquid may be blood. Once expanded, absorbent layer 416 may be soft and pliable. Without being limited to any particular theory, this quality may permit absorbent layer 416 to conform to irregular wound crevices, gaps, and fissures.

According to various embodiments, an expanded absorbent layer 416 occupies a volume greater than an unexpanded absorbent layer 416. In various embodiments, the average volume ratio of the expanded layer to the unexpanded layer is at least 2× (in other words, the volume of the expanded layer is capable of expanding to at least 2 times the volume of the unexpanded layer 416). In other embodiments, the average volume ratio of the expanded layer to the unexpanded layer is at least 4×. In other embodiments, the average volume ratio of the expanded layer to the unexpanded layer is at least 8×. In other embodiments, the average volume ratio of the expanded layer to the unexpanded layer is at least 10×.

In various embodiments, the wound-contacting absorbent layer 416 may be capable of expanding to 80% or greater of its maximum expansion capacity in 60 seconds or less following immersion in liquid, for example in 30 seconds or less, in 10 seconds or less, or in 5 seconds or less following immersion in liquid. In certain environments, in use, the wound-contacting absorbent layer 416 may be unable to reach its maximum expansion capacity, as it may be constrained in some manner, such as by the dimensions of the wound.

In various embodiments, absorbent layer 416 comprises an absorbent material including, but not limited to, a sponge or fibrous material. In various embodiments, the absorbent material may comprise a polysaccharide such as, but not limited to, cellulose, starch, chitin, or chitosan. In an embodiment, absorbent layer 416 comprises regenerated cellulose sponge. In other embodiments, the absorbent material may comprise synthetic sponges such as, but not limited to, various polyvinyl alcohol (PVA) polymers and derivatives thereof having desirable physical and mechanical properties. In various embodiments, absorbent layer 416 may be biodegradable and/or bioabsorbable.

In various embodiments, the absorbent material may comprise a contiguous sheet. In other embodiments, the absorbent material may comprise a plurality of distinct articles that move relatively independent of one another.

In various embodiments, absorbent layer 416 may comprise a compressed material. A “compressed material” refers to a material that has a first size/dimension or form factor in a non-compressed state, and has a second, reduced size/dimension or form factor in a compressed state. In various embodiments, the compressed material may remain in essentially a compressed state indefinitely until hydrated. For these embodiments, and without being limited to any particular theory, the compressed material, when hydrated, may rapidly expand in an effort to assume its pre-compression dimensions. In this way, the compressed material may store additional mechanical energy in a compressed state, as compared to the non-compressed state, that is released when exposed to a liquid, thus causing absorbent layer 416 to quickly expand. The absorbent material can be compressed by heat compression or any other suitable compression method known in the art.

FIGS. 4A and 4B also illustrate a compression bandage with a pressure indicator 418 in an “unpopped” state (FIG. 4A) and a “popped” state (FIG. 4B), in accordance with various embodiments. In various embodiments, the pressure indicator 418 may take any of several different forms, such as a valve, sensor, etc. In various embodiments, the pressure indicator 418 may be in fluid communication with the air bladder 404 to provide an indication of the current pressure in the air bladder 404 and/or to provide an indication that a threshold pressure has been exceeded.

In some embodiments, a pressure indicator 418 may be configured to mechanically respond to pressure in the air bladder 404 when it exceeds a predetermined pressure threshold, wherein the pressure indicator 418 exhibits a first state or conformation below the threshold (e.g., as shown in FIG. 4A) and a second state or conformation above the threshold (e.g., as shown in FIG. 4B). In various embodiments, such a pressure indicator 418 may remain in the second state or conformation when the pressure falls below the threshold, or, alternatively, the pressure indicator 418 may return to the first state or conformation when the pressure falls below the threshold. In some embodiments, two or more pressure indicators 418 may be provided that respond to different thresholds.

As an example, in various embodiments, a pressure indicator 418 may be a visual indicator bubble that may pop out of the air bladder 404, such as in a dome shape, when a desired inflation pressure has been reached. In one embodiment, the pop up pressure threshold may be 400 mmHg. In various embodiments, such a pop-up pressure indicator 418 may be adjusted to pop out at different predetermined pressures, such as from 200 mmHg to 600 mmHg, for example by altering the shape of the bubble, the diameter of the bubble, the thickness of the material, and/or the type of material used to construct the bubble. In some embodiments, a pop-up pressure indicator 418 may have a first state or conformation in which the bubble is flat, depressed, or deflated, and a second state or conformation in which the bubble is extended outward, resulting from the pressure in the air bladder 404 exceeding the predetermined threshold pressure.

In various embodiments, the pressure indicator 418 may be mounted directly on the air bladder 404, or in any location that is in communication with the fluid used to expand the air bladder 404.

Various embodiments provide a film-based pressure indicator 418, primarily including a single-layer thermoplastic material. Such an embodiment may be constructed with many optional materials and configurations, which will be discussed below as reference examples only. Without being bound by theory, the pressure indicator 418 may be based on a predetermined interruption of a standard surface. The interruption in surface integrity may react differently than adjacent areas, when it is exposed to an increase of fluid pressure.

In various embodiments, a pressure indicator 418 may be formed using a variety of techniques. In one such embodiment, vacuum forming of pre-heated thermoplastic films into or over a variety of shapes may be used for creating fixed geometries in various thermoplastic materials. In a specific embodiment, the thermoplastic film may be heated to its softening point (melting point) and then drawn into or over a shape, where vacuum is applied to further enhance the movement of the film into intimate contact with the desired forming shape or mold. The thermoplastic film cools (crystalizes) to the precise shape of the mold. Once the film has cooled sufficiently, the vacuum is released and the thermoplastic film is transformed into a replication of the mold to which it was applied.

In one specific example, a thermoplastic polyurethane film at 0.030 inch thickness and a hardness of approximately 87 Shore ‘A’ is heated to its softening point (melting point) of approximately 340° F.-380° F. Depending on the heat source applied to soften/melt the film, it may take several seconds to achieve the desired melting temperature. Once the film is at the correct forming temperature, it is then drawn into a forming mold/tool with vacuum assist. The forming mold/tool may have a plurality of forming cavities, engineered to create specific geometric shapes in the thermoplastic film. This is achieved following a sufficient cooling cycle against the forming surface. Specifically, in some embodiments, the mold/tool has concave hemispheric shapes approximately 0.500 inch diameter and a depth of approximately 0.250 inch. In various embodiments, the hemispheres may be vented to allow full evacuation of air volume and to support accurate shape replication when the thermoplastic film is vacuum drawn into the hemispheres. In various embodiments, the mold/tool may be fabricated of heat resistant materials such as aluminum, or brass. In various embodiments, the mold/tool may also be fabricated using laser sintering to create a fully porous structure.

In various embodiments, the newly-formed thermoplastic film hemispheres may then be cut with a circular cutting die to relieve them from the master forming sheet film surface. This cut may be extended beyond the hemisphere perimeter to achieve a final cut perimeter of approximately 1.000 inch diameter. In various embodiments, a single film hemisphere is then fitted into a pre-cut hole approximately 0.750 inch diameter which is created in a first layer thermoplastic film sheet 0.012 inch thick. This same film sheet may also have at least one other pre-cut hole that accepts a thermoplastic injection molded fitment. In various embodiments, the injection molded fitment may later act as a port to introduce fluid into a perimeter sealed thermoplastic vessel/bladder.

In various embodiments, a hollow circular brass sealing tool may then be positioned on top of the film hemisphere outer perimeter. In various embodiments, the film hemisphere may then be positioned and trapped firmly against the first layer film sheet. The hollow circular brass sealing tool may have a sealing width of approximately 0.125 inch in various embodiments. Pressure and energy may be introduced to the brass sealing tool to create sufficient surface contact and internal heat to melt the perimeter of the film hemisphere to the adjacent first layer film sheet. In an embodiment, radio frequency welding also may be used as a suitable sealing methodology. In various embodiments, a slight cooling cycle may be employed to ensure thermoplastic crystallization before releasing pressure on the brass sealing tool. In various embodiments, the film hemisphere may now be permanently attached to the first layer film sheet. This same general procedure may be used for any additional thermoplastic fitments added to the first layer film sheet, such as an injection molded fluid port.

In various embodiments, second layer film sheet is now positioned to the opposite side of the first film sheet such that first film sheet and its accompanying fitments are not covered by the second film sheet. In an embodiment, a primarily square hollow brass sealing die approximately 5.000 inches with hemispherical and rectangular extensions may be positioned on top of the two layers of thermoplastic film. In various embodiments, the hollow brass sealing tool may have a sealing width of approximately 0.200 inch. Pressure and energy may be introduced to the brass sealing tool to create sufficient surface contact and internal heat to melt the perimeter of the film sheets together. In an embodiment, radio frequency welding may be used as the selected sealing methodology. A slight cooling cycle may then be employed to ensure thermoplastic crystallization before releasing pressure on the brass sealing tool. In various embodiments, the first and second thermoplastic film layers may now be permanently attached along the perimeter. Excess film from the first and second film sheets may be cut away along the outer edge of the perimeter seal, leaving a clean sealed perimeter margin.

In various embodiments, a fitment tube may now be introduced and attached to the injection molded fluid port on the thermoplastic film vessel/bladder. In various embodiments, fluid may be introduced into the fitment tube and may transfer directly into the thermoplastic film vessel/bladder. In various embodiments, as the fluid pressure increases, the thermoplastic film vessel/bladder may expand. Additionally, the fluid pressure may act upon the inside surface of the film hemisphere. In various embodiments, as the internal pressure increases, the film hemisphere may mobilize and invert/pop outward. In various embodiments, the distinct inversion in the film hemisphere may indicate that a predetermined fluid pressure threshold has been achieved.

In various embodiments, the film hemisphere may provide a visual, and possibly audible, indication of internal fluid pressure within the thermoplastic film vessel/bladder. Such an audible indication may be from the movement of the film (or other pressure indicator), or by triggering a separate audible signal.

In an embodiment, a pressure indicator may be a tunable measurement system. It should be noted that variations in film thickness, film hardness, and geometry may all contribute to the movement of a pressure indicator. It should also be noted that engineered injection molded thermoplastic geometries may also act similarly as low cost, thin wall pressure indictors when the descriptive techniques enclosed herein are applied.

In various embodiments, a pressure indicator may be employed alone or within a group setting to achieve a single fluid pressure reading or multiple and varied fluid pressure readings. In various embodiments, a series of pressure indicators may be arrayed in a group with each separate indicator calibrated to “pop” at a different bladder pressure (or otherwise indicate the threshold has been reached). In this configuration, the series of indicators may act as a pressure gauge. For example if two or more film hemispheres with dissimilar diameters are used on a single thermoplastic film vessel/bladder, it is possible to have multiple pressure indications based on variations in fluid pressure required to invert a given thermoplastic film hemisphere geometry.

In various embodiments, a pressure indicator also may have a stepped film hemispherical shape that inverts in a predetermined sequence, with larger steps ‘popping’ before smaller steps. In various embodiments, the stepped film hemisphere may indicate variations in internal fluid pressure within a given thermoplastic vessel/bladder. In various embodiments, other pressure indicators may activate a pulse type switch, for example to illuminate a warning light or audible signal. In various embodiments, a film hemisphere upon inversion may force against another surface, which in turn may activate an optional signal mechanism.

In various embodiments, a pressure indicator may be colored differently from the surrounding thermoplastic film vessel/bladder. In some embodiments, a discrete third layer of thermoplastic film may be applied and permanently affixed directly above the film hemisphere. In various embodiments, the third layer of film may have a thin slot in its surface directly above the film hemisphere. Thus, as fluid pressure increases on the inside surface of the film hemisphere, it may invert/pop through the slot in the third layer film surface and expose a unique color warning when desired fluid pressure has been achieved.

In various embodiments, a pressure indicator and accompanying thermoplastic vessel bladder may be constructed with other readily available forming techniques such as heat sealing, ultrasonic welding, and adhesives. In various embodiments, a variety for thermoplastic resins may be used to create a pressure indicator and/or a thermoplastic vessel/bladder, such as polyurethane, polypropylene, polyethylene, nylon, PVC, EVA, etc.

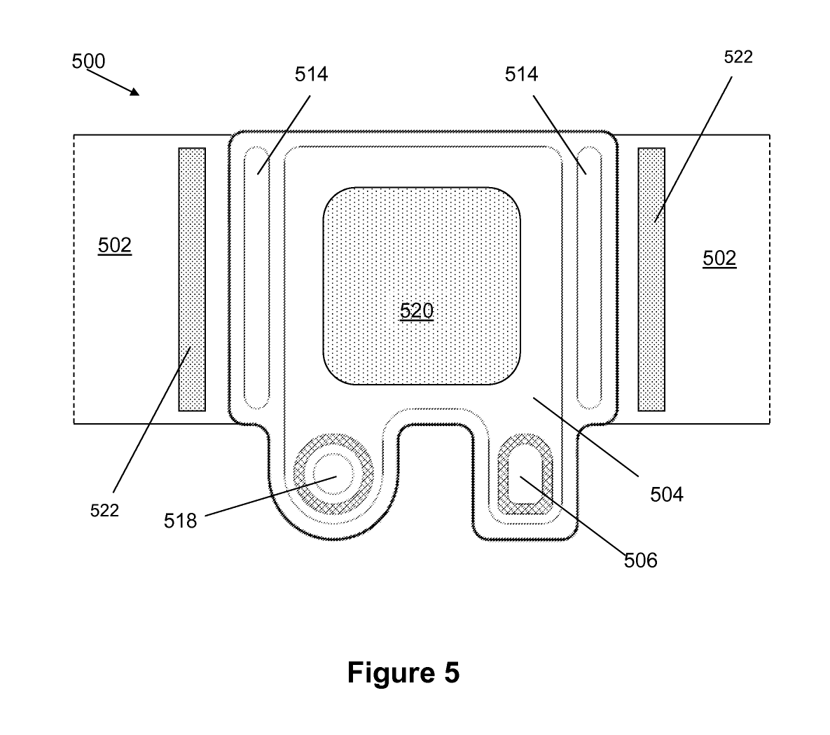

FIG. 5 is a diagram of another example of a compression bandage 500 with an air bladder 504 having a pressure indicator 518 and an inflation mechanism 506, for use in accordance with various embodiments. In the illustrated embodiment, stitching margins 514 are included on the outer left and right sides of air bladder 504. In various embodiments, such stitching margins may be used to stitch or otherwise couple the air bladder 504 to the bandage 502. Additionally, the illustrated embodiment includes a slip-resistant material 520 disposed on an outer surface of air bladder 504 to prevent bandage 502 from accidentally slipping off air bladder 504 when the bandage 502 is wrapped around a wound site, such as around an arm, leg, torso, etc. In various embodiments, the slip-resistant material 520 may include a continuous slip-resistant surface, or it may be arranged in spots, patches or a plurality of lines or other shapes. In one specific embodiment, the slip-resistant material 520 may include a plurality of barbs, such as a hook and loop fastener (such as VELCRO®). In other embodiments, the slip-resistant material 520 may include a rubber-like coating, adhesive, or tape.

In other embodiments, one or more non-slip strips 522 may be included on the bandage 502, for instance on either side of air bladder 504. Like the slip-resistant material 520 that may be coupled to the air bladder, 504, the one or more non-slip strips 522 may help prevent the bandage 502 from accidentally slipping off air bladder 504 when the bandage 502 is wrapped around a wound site, such as around an arm, leg, torso, etc. In various embodiments, the one or more non-slip strips 522 may include a continuous slip-resistant surface, or they may be arranged in spots, patches or a plurality of lines or other shapes. In one specific embodiment, the one or more non-slip strips 522 may include a plurality of barbs, such as a hook and loop fastener (such as VELCRO®). In other embodiments, the one or more non-slip strips 522 may include a rubber-like coating, adhesive, or tape. In various embodiments, non-slip strips 522 may be positioned on the bandage at an edge-to-edge distance of 0-10 cm from the air bladder. In other embodiments, the non-slip strips 522 may be positioned on the bandage at an edge-to-edge distance of 1-5 cm from the air bladder.

In another embodiment, there is provided a method to effect rapid hemostatic response and hemorrhage control by applying a pneumatic compression bandage as described herein to a bleeding wound and inflating the air bladder to provide a therapeutic effect. In various embodiments, applying a pneumatic compression bandage to a wound may include applying the compression bandage by hand. In some embodiments, the compression bandage may be applied onto a wound using a securing adhesive. In another embodiment, the compression bandage may be wrapped around a wound site, such as around an arm, leg, torso, etc.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

We claim:

1. A pneumatic compression bandage, comprising:
a bandage; and
a fluid bladder disposed on the bandage, wherein the fluid bladder comprises:
an inflation mechanism for selectably inflating the fluid bladder; and
a pressure indicator, wherein the pressure indicator provides an indication of the pressure within the fluid bladder.

2. The pneumatic compression bandage of claim 1, wherein the inflation mechanism comprises a manually-operable inflation mechanism.

3. The pneumatic compression bandage of claim 1, wherein the pressure indicator undergoes a conformational change when the pressure in the fluid bladder exceeds a predetermined threshold, and wherein the conformational change comprises a transition from a first conformation when the pressure is below the predetermined threshold to a second conformation when the pressure exceeds the predetermined threshold.

4. The pneumatic compression bandage of claim 3, wherein the pressure indicator comprises an invertible dome.

5. The pneumatic compression bandage of claim 1, wherein the bandage comprises an elastic bandage.

6. The pneumatic compression bandage of claim 1, wherein the bandage further comprises an absorbent layer.

7. The pneumatic compression bandage of claim 6, wherein the absorbent layer expands in occupied volume upon contact with a liquid.

8. The pneumatic compression bandage of claim 6, wherein the bandage comprises a first side and a second side, wherein the air bladder is disposed on the first side, and wherein the absorbent layer is disposed on the second side, opposite the air bladder.

9. The pneumatic compression bandage of claim 1, wherein the fluid bladder comprises a non-slip surface.

10. A pneumatic compression bandage, comprising:
a bandage;
a fluid bladder disposed on the bandage;
an inflation mechanism for selectably inflating the fluid bladder; and
a non-slip material disposed on at least one side of the air bladder.

11. The pneumatic compression bandage of claim 10, wherein the non-slip material comprises hook-and-loop fastening elements.

12. The pneumatic compression bandage of claim 10, wherein the bandage comprises an elastic bandage.

13. The pneumatic compression bandage of claim 10, wherein the bandage further comprises an absorbent layer.

14. The pneumatic compression bandage of claim 13, wherein the absorbent layer expands in occupied volume upon contact with a liquid.

15. The pneumatic compression bandage of claim 13, wherein the bandage comprises a first side and a second side, wherein the air bladder is disposed on the first side, and wherein the absorbent layer is disposed on the second side, opposite the air bladder.

16. The pneumatic compression bandage of claim 10, wherein the non-slip material is positioned on the bandage at an edge-to-edge distance of 0-10 cm from the air bladder.

17. The pneumatic compression bandage of claim 10, wherein the non-slip material is positioned on the bandage at an edge-to-edge distance of 1-5 cm from the air bladder.

18. A pressure indicator, comprising an invertible dome, wherein the invertible dome undergoes a conformational change when the pressure in the invertible dome exceeds a predetermined threshold, and wherein the conformational change comprises a transition from a first conformation when the pressure is below the predetermined threshold to a second conformation when the pressure exceeds the predetermined threshold.

19. The pressure indicator of claim 18, wherein the invertible dome comprises one or more steps, and wherein each step inverts in response to a different pressure.

20. The pressure indicator of claim 18, wherein the pressure indicator comprises a plurality of invertible domes each with a different pressure threshold.
